(19) United States
(12) Patent Application Publication
(10) Pub. No.: US 2012/0071352 A1
(43) Pub. Date: Mar. 22, 2012

(54) Non-labeled virus detection substrate, system, and method based on inverted multi-angular cavity arrays

(75) Inventors: Jiunn-Der Liao, Tainan City (TW); Chia-Wei Chang, Tainan City (TW); Chih-Kai Yao, Tainan City (TW)

(73) Assignee: National Cheng Kung University, Tainan City (TW)

(21) Appl. No.: 13/137,710

(22) Filed: Sep. 7, 2011

Related U.S. Application Data
(60) Provisional application No. 61/380,413, filed on Sep. 7, 2010.

(51) Int. Cl. C40B 30/10; C40B 60/12; C12M 1/00

(52) U.S. Cl. 506/12; 435/283.1; 506/39

(57) ABSTRACT

The present invention discloses a non-labeled virus detection substrate, system, and method based on inverted multi-angular cavity arrays. The virus detection substrate is used together with a Raman spectrometer for virus detection. The virus detection substrate has a metal layer disposed thereon, and inverted multi-angular cavities are formed in the metal layer. The cavities are arranged in a microarray. In order to detect the target, the size of the cavities should be adjusted first. Then, a laser with an optimized wavelength is applied to induce the effect of the surface enhanced Raman scattering.

### CROSS REFERENCE TO RELATED APPLICATION

This application claims the benefit of filing date of U.S. Provisional Application Ser. No. 61/380,413, entitled “Non-labeled virus detection using inverted triangular Au nano-cavities arrayed as SERS-active substrate” filed Sep. 7, 2010 under 35 USC §119(e)(1).

### BACKGROUND OF THE INVENTION

1. Field of the Invention

The present invention relates to a virus detection substrate, system, and method and, more particularly, to a virus detection substrate, system, and method suitable to be performed together with a Raman spectrometer.

2. Description of Related Art

Rapid and accurate detection and identification of a dangerous pathogen is extremely helpful for a diagnosis or therapy of an early-stage disease caused by the pathogen. Currently, antibody-based bioarrays can be employed to monitor viruses and such techniques are, for example, enzyme-linked immunosorbent assays (ELISA), fluorescent antibody arrays, and serological testing. However, these techniques may meet some clinical problem such as being time-consuming, false positive, etc.

In recent years, as polymerase chain reaction (PCR) keeps advancing, related methods such as single-nucleotide polymorphism (SNP) in which a target DNA sequence is also proliferated, become alternative approaches to virus identification. However, complex purification and isolation steps of the target DNA are required to be performed prior to ELISA or PCR. Therefore, if such complex purification and isolation steps can be eliminated, identification of a target virus can be simplified and accelerated.

At present, in virus identification or detection, the major difficulties are associated with the complex purification and isolation, the detectable amount, apparent size, and dimension of virus, and the requirement of chemical identification databases for specific viruses. In general, rapid-screening detection is difficult to achieve at an early stage of virus infection since the virus amount often is unable to reach the detectable amount, more than 10⁶ plaque-forming units (PFU)/ml, in biomolecular assays where the purification procedure is employed. The reagents used in common biomolecular assays are far smaller than an apparent size and dimension of a virus. Thus, only a portion of the integral virus can be detected, resulting in incomplete virus information. Also, it is difficult to establish chemical identification information for the integral virus in a database.

Biomolecules or their fragments can be investigated by resonating mechanical cantilevers, evanescent wave biosensors, or atomic force microscopy, but these approaches are used only to measure the amount of the biomolecules. Alternatively, a labeling method can be employed to detect microorganisms by a particular functional group. However, it is difficult to prepare samples without contamination, or easy to overlap between signals of the labeling functional group and the target microorganism. Hence, these techniques consume a lot of time for detection and can not meet a clinical requirement for rapid detection.

### SUMMARY OF THE INVENTION

The object of the present invention is to provide a virus detection substrate, system, and method. In the virus detection substrate, inverted multi-angular nano-cavities are formed and arranged in an array. It can be used to increase the cross-section area of Raman scattering. Then, the surface enhanced Raman scattering (SERS) effect would be induced, so as to accelerate preparation of a sample, determine the actual structure of a specific virus, and construct chemical identification information for viruses.

In order to achieve the object mentioned above, the present invention provides a virus detection substrate used together with a Raman spectrometer for virus detection, and the virus detection substrate includes a metal layer disposed thereon and multiple cavities are formed in the metal layer and arranged in a microarray. For example, the cavities are arranged in an array to form a microstructure.

In addition, the present invention also provides a virus detection system used to detect a virus sample. The virus detection system includes: a virus detection substrate used to hold the virus sample and comprising a metal layer disposed thereon, wherein multiple cavities are formed in the metal layer and arranged in a microarray; a Raman spectrometer applying an incident laser onto the metal layer of the virus detection substrate to obtain a Raman scattering signal; and a receiver device used to receive the Raman scattering signal and output a Raman spectrum.

Moreover, the present invention further provides a method for detecting viruses including the following steps: providing a virus detection substrate and a Raman spectra virus database, wherein the virus detection substrate comprises a metal layer disposed thereon and multiple cavities are formed in the metal layer and arranged in a microarray; dropping a virus sample onto the inner surface of the cavities of the virus detection substrate; applying an incident light by a Raman spectrometer onto the metal layer of the virus detection substrate to generate a Raman spectrum of the virus sample; and comparing the Raman spectrum of the virus sample with the Raman spectra virus database to identify the species of the virus sample.

In the present invention, the metal layer can be made of one selected from the group consisting of gold, silver, copper, and an alloy thereof. In one aspect of the virus detection substrate, the metal layer is made of gold.

In addition, the cavities can be formed in a shape of an inverted multi-angular pyramid. The distance between the tips of every two of the cavities can be in a range from 100 nm to 1000 nm, and preferably is 500 nm. The depth of each of the cavities can be in a range from 50 nm to 300 nm, and preferably is in a range from 90 nm to 120 nm. In the present invention, the depth of the cavities each shaped with an inverted multi-angular pyramid needs to be adjusted according to the size (diameter) of the virus to be tested. If the dimensions of the cavities corresponding to the size of the virus are selected, the optimized surface-enhanced Raman scattering effect can be determined. Furthermore, the wavelength of the incident laser mentioned above also needs to be regulated according to the size of the cavities, so as to ensure obtaining the optimized signal of the surface-enhanced Raman scattering effect.

In the present invention, the species of the detectable virus is not particularly limited and can be, for example, encephalomyocarditis virus (EMCV), adenovirus, and influenza A virus. Among these viruses, EMCV is a non-enveloped positive-sense RNA virus in a diameter of about 30 nm, and able to infect vertebrates. Adenovirus is a non-enveloped double-stained linear DNA virus in a diameter of about 80-90 nm, and able to infect birds and mammals. Influenza A virus is an enveloped negative-sense segmented RNA virus in a diameter of about 90-120 nm. At present, based on the hemagglutinin (HA) and neuraminidase (NA) on the envelope, influenza A virus can be classified into sixteen HA subtypes (i.e. H1 to H16) and nine NA subtypes (i.e. N1 to N9).

As mentioned above, various viruses possess different sizes and specific characteristics. However, conventional analyses for viruses are time-consuming. When infective viruses such as influenza A virus cause large-scale epidemics, there is an urgent need for a rapid and accurate virus detection system or method. Once the viruses are identified, the subsequent therapy can be executed for rapid control over viral epidemics.

For rapid detection of viruses, the virus detection substrate, system, and method of the present invention are accomplished. In the present invention, the nano-mechanical method is employed with various indentation tips to form dense cavities each shaped in an inverted multi-angular pyramid and arranged in an array. Thus, contamination on the surface of the substrate can be avoided and SERS effects during virus detection can be enhanced. Even if viruses are in a small amount, the viruses can be identified according to enhanced SERS spectra to give rapid detection of viruses.

### BRIEF DESCRIPTION OF THE DRAWINGS

FIG. 1A shows a top view of a cavity arrangement on the virus detection substrate in the example of the present invention;

FIG. 1B shows the distance between two of the cavities arranged on the virus detection substrate in the example of the present invention, wherein the distance is represented as Dₜ₋ₜ;

FIG. 1C shows the depth of each of the cavities arranged on the virus detection substrate in the example of the present invention, wherein the depth of the cavities is a sum of hₚ₋ᵤ and hₛ₋ᵢ; and represented as Dᵥ, and hₚ₋ᵤ and hₛ₋ᵢ respectively denotes the vertical distances from the top edge and the bottom tip of the cavities to the surface of the metal layer;

FIG. 2 shows a field emission scanning electron microscopy (FE-SEM) photograph of the virus detection substrate in the example of the present invention, wherein (A) shows substrate No. 1 with 1000/50 of Dₜ₋ₜ/Dᵥ; (B) shows substrate No. 2 with 1000/70 of Dₜ₋ₜ/Dᵥ; (C) shows substrate No. 3 with 1000/90 of Dₜ₋ₜ/Dᵥ; (D) shows substrate No. 4 with 1000/120 of Dₜ₋ₜ/Dᵥ; (E) shows substrate No. 5 with 1000/140 of Dₜ₋ₜ/Dᵥ; (F) shows substrate No. 6 with 500/50 of Dₜ₋ₜ/Dᵥ; (G) shows substrate No. 7 with 500/70 of Dₜ₋ₜ/Dᵥ; (H) shows substrate No. 8 with 500/90 of Dₜ₋ₜ/Dᵥ; (I) advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example

### A. Preparation of Virus Detection Substrate

A polished single crystal silicon (100) wafer (Silicon Sense, Germany) was prepared first, and primed with a titanium adhesion layer in a thickness of about 5 nm. On the titanium adhesion layer, a gold layer (99.99% purity) was deposited in a thickness of about 200 nm by thermal evaporation to give a gold substrate. The grain size and root mean squared roughness of the gold substrate were measured with a scanning probe microscope (SPA300HV, SEIKO, Japan). The result shows the deposited gold consists of polycrystalline grains in a size from 20 to 50 nm and the grains were arranged in an orientation of (111). In addition, the root mean squared roughness was about 0.62 nm.

Under a controlled relative humidity (about 32%) and room shows substrate No. 9 with 500/120 of Dₜ₋ₜ/Dᵥ; and (J) shows substrate No. 10 with 500/140 of Dₜ₋ₜ/Dᵥ;

FIG. 3 shows an SERS spectrum and its histogram of peak intensity for R6G detection with the virus detection substrate in the example of the present invention;

FIG. 4 shows an SERS spectrum for EMCV detection with the virus detection substrate in the example of the present invention, wherein (A) denotes EMCV in a concentration of 10⁷ PFU/mL; and (B) denotes EMCV in a concentration of 10⁶ PFU/mL;

FIG. 5 shows an SERS spectrum for adenovirus detection with the virus detection substrate in the example of the present invention, wherein (A) denotes adenovirus in a concentration of 10⁷ PFU/mL; and (B) denotes adenovirus in a concentration of 10⁶ PFU/mL;

FIG. 6 shows an SERS spectrum for influenza A virus detection with the virus detection substrate in the example of the present invention, wherein (A) denotes influenza A virus in a concentration of 10⁷ PFU/mL; and (B) denotes influenza A virus in a concentration of 10⁶ PFU/mL; and

FIG. 7 shows FE-SEM photographs for virus detection with the virus detection substrate in the example of the present invention, wherein (A) denotes EMCV; (B) denotes adenovirus; and (C) denotes influenza A virus.

### DETAILED DESCRIPTION OF THE PREFERRED EMBODIMENT

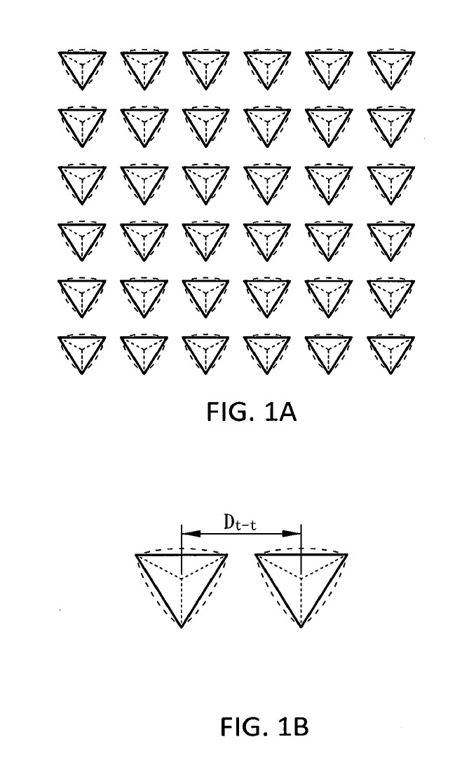

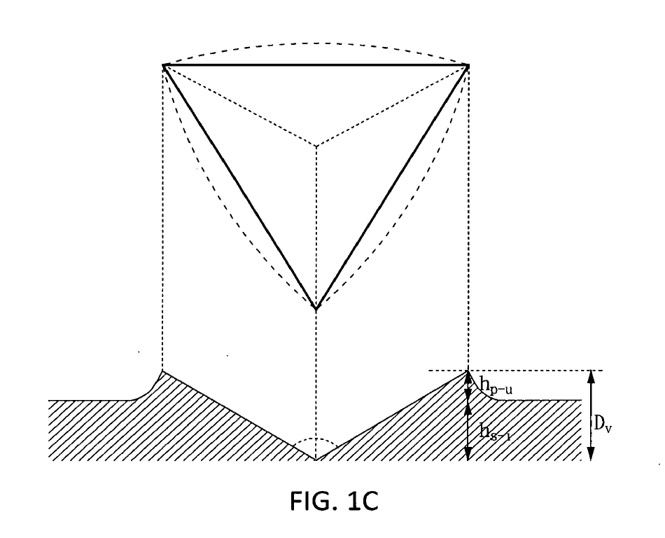

Because of the specific embodiments illustrating the practice of the present invention, one skilled in the art can easily understand other temperature (about 24° C.), the gold substrate was indented by dynamic contact module system (NanoIndenter G200, Agilent Technologies, USA) with triangular pyramid nano-indentation tips of Berkovich diamond (in a radius of about 20 nm) in one-step loading-unloading mode to form cavities. Prior to indentation, the nano-indentation tips were cleaned by indenting on single crystal aluminum. The loading procedure was controlled at a constant drift rate of 0.05 nm/sec. The loading force was applied in the range from 270 to 1700 μN depending on Dᵥ of 50, 70, 90, 120, and 140 nm measured by vertical displacement. Dₜ₋ₜ was fixed in 500 and 1000 nm. D, and Dₜ₋ₜ were two major geometrical factors. As shown in FIG. 1A, the formed cavities are arranged in an array of 6×6. However, the array arrangment of the present invention is not limited thereto and can also be 7×7 or 10×10. As shown in FIG. 1B, the distance (Dₜ₋ₜ) between two cavities indicates the horizontal distance between the bottom tips of two cavities. As shown in FIG. 1C, some material of the substrate is squeezed out the cavities and piles up therearround during the indentation. Accordingly, the height from the substrate surface to the top of the piling-up material is denoted as hₚᵢₗₑ₋ᵤₚ (hₚ₋ᵤ), and the depth from the substrate surface to the bottom tips of the cavities is denoted as hₛᵢₙₖ₋ᵢₙ (hₛ₋ᵢ). Therefore, the composite microstructure is created simultaneously with sinking and protrusion, and the integral depth of the cavities (Dᵥ) means the vertical distance in a sum of hₚ₋ᵤ and hₛ₋ᵢ. In addition, the approaching velocity and the harmonic displacement of the nano-indentation tips toward the surface of the gold substrate were maintained at 1 nm/sec and 1 nm. Depending on the unspecified contact points, surface approaching sensitivity was optimized at 10% for the gold substrate.

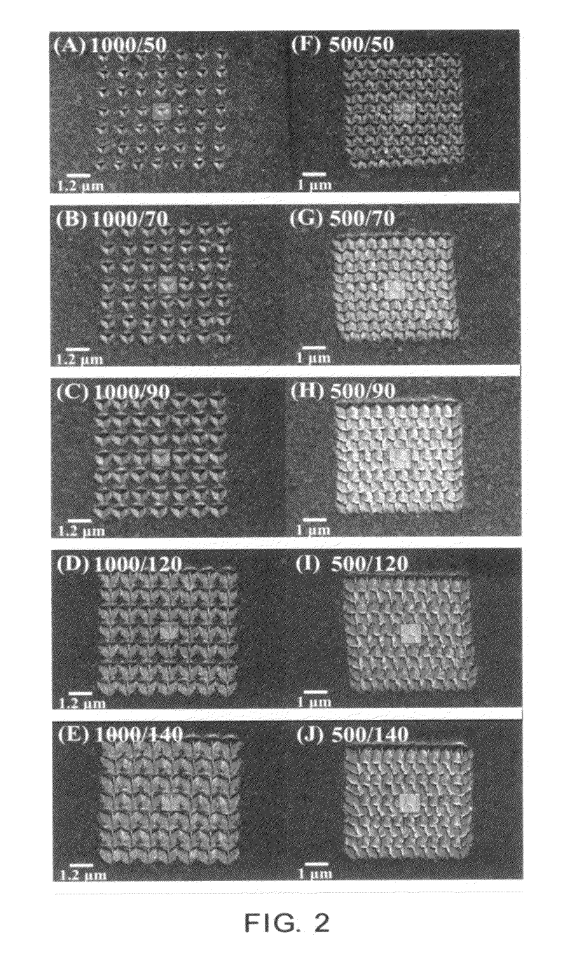

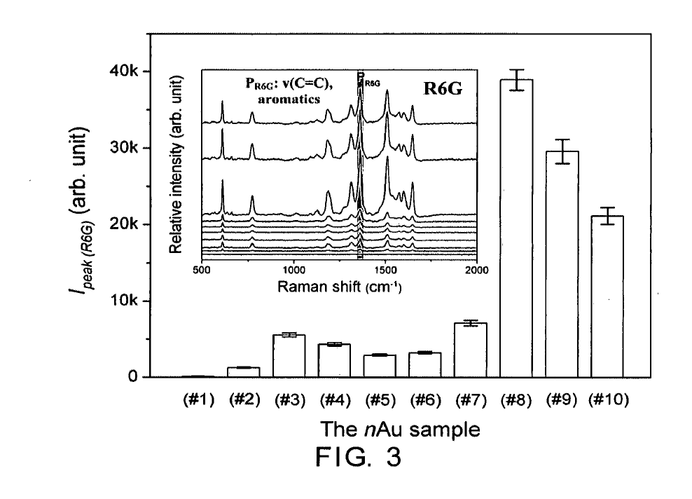

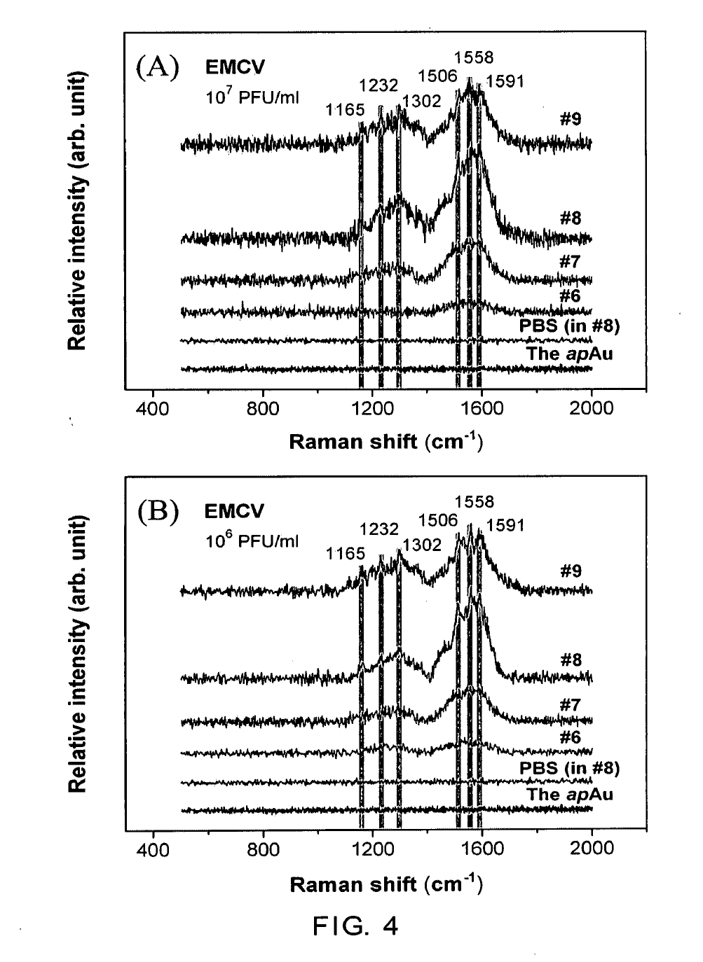

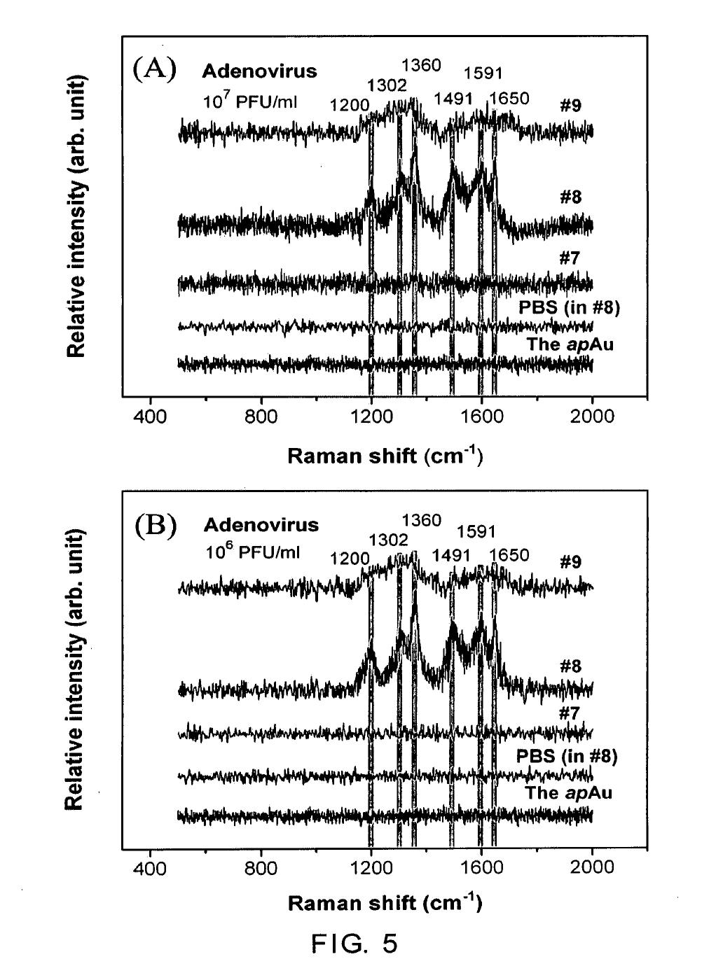

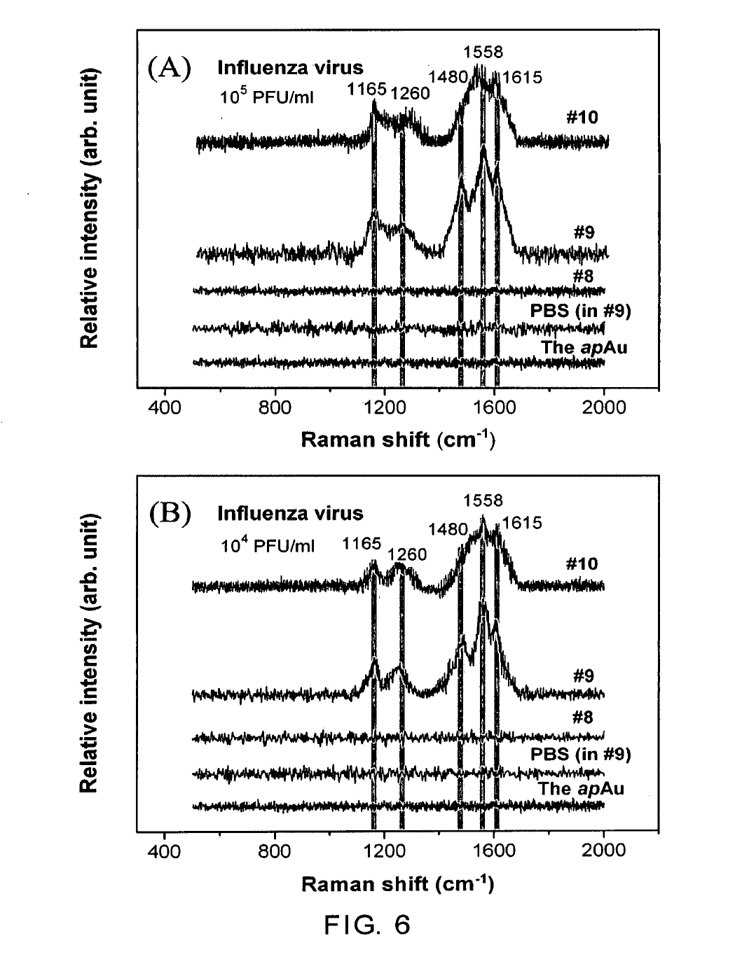

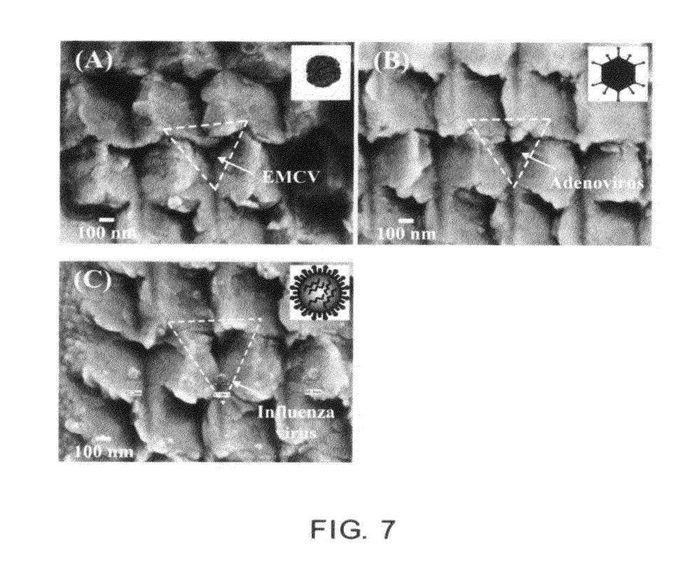

The virus detection substrates were manufactured according to Dᵥ (50, 70, 90, 120, and 140 nm) and Dₜ₋ₜ (500 and 1000 nm) and observed with a field emission scanning electron microscopy (FE-SEM). The resultant FE-SEM photographs are shown in FIG. 2. In FIG. 2, (A) shows substrate No. 1 with 1000/50 of Dₜ₋ₜ/Dv; (B) shows substrate No. 2 with 1000/70 of Dₜ₋ₜ/Dv; (C) shows substrate No. 3 with 1000/90 of Dₜ₋ₜ/Dv; (D) shows substrate No. 4 with 1000/120 of Dₜ₋ₜ/Dv; (E) shows substrate No. 5 with 1000/140 of Dₜ₋ₜ/Dv; (F) shows substrate No. 6 with 500/50 of Dₜ₋ₜ/Dv; (G) shows substrate No. 7 with 500/70 of Dₜ₋ₜ/Dv; (H) shows substrate No. 8 with 500/90 of Dₜ₋ₜ/Dv; (I) shows substrate No. 9 with 500/120 of Dₜ₋ₜ/Dv, and (J) shows substrate No. 10 with 500/140 of Dₜ₋ₜ/Dv.

### B. Test for SERS Effects

The R6G molecule is commonly used as a tracer dye and extensively applied in biotechnology applications such as fluorescence microscopy, flow cytometry, and enzyme-linked immunosorbent assays (ELISA). Herein, the R6G molecule was diluted with phosphate-buffered saline (PBS, Sigma, Germany) to form a R6G solution in a concentration of 10⁻⁴ M. The R6G solution was pipetted on the surfaces of the substrates Nos.1 to 10, covered with a slide, and then immediately measured by Raman spectroscopy. In the Raman spectroscopy, the R6G molecule was used as a probe to recognize SERS effects of the gold substrates, and He—Ne laser was used and controlled in a wavelength of 633 nm to give enhanced effects for R6G detection.

The results are shown in FIG. 3. In FIG. 3, #1 to #10 respectively represent the substrates Nos. 1 to 10 described above. The major peak of the R6G molecule designated “P_{R6G}”, is clearly found at 1360 cm⁻¹ (v(C=C), aromatics). Raman intensity of “P_{R6G}” is denoted as I_{peak(R6G)}. Based on FIG. 3, the substrates #6 to #10 show preferable intensity of SERS effects. The result indicates that Raman intensity is significantly increased in the substrates with 500 nm of Dₜ₋ₜ. In addition, Dᵥ and Dₜ₋ₜ are capable of regulating the resonance frequency of localized surface plasmon so that the resonance frequency can conform to an incident laser wavelength and the induction of electromagnetic (EM) effects can be maximized by the nano-structures.

### C. Test for Virus Detection

Encephalomyocarditis virus (EMCV), adenovirus, and influenza A virus were prepared. EMCV was propagated in Vero cells (African green monkey kidney cells). An adenovirus vector derived from wild-type adenovirus type 5, which was defective in E1B-55 kD gene, was transfected in 293 cells (human embryonic kidney cells). Influenza A virus/WSN/33 (H1N1) was propagated in MDCK cells (Madin-Darby canine kidney cells).

All of the cells were cultured in Dulbecco's modified Eagles medium (DMEM) with 10% cosmic calf serum (Hyclone, USA). Standard protocols were used to propagate different viruses. The titers of the viruses were determined by the plaque assay. The results showed that titers of EMCV, adenovirus, and influenza A virus were 10⁸, 10⁸, and 10⁶ plaque-forming unit (PFU), respectively. EMCV and adenovirus were diluted with PBS to form the titers 10⁷ and 10⁶ PFU/ml, and influenza A virus was diluted with PBS to form the titers 10⁴ and 10⁵ PFU/ml.

The diluted viruses were pipetted in 5 μl on the virus detection substrates mentioned above and examined with a confocal microscopic Raman spectrometer (In Via Raman microscope, RENISHAW, United Kingdom) using He—Ne laser at 633 nm with the power of 17 mW. The scattering light was collected by a 50× objective lens to a charge-coupled device (CCD) detector. A grating of 1800 lines/mm was used to disperse the scattered light. All the reported Raman spectra were the results of 10-seconds accumulation in a single time in a range of 500-2000 cm⁻¹. Prior to each test, Raman shifts were normalized using the signal of 520 cm⁻¹ with the same absolute intensity from a standard silicon wafer.

With reference to FIGS. 4(A) and 4(B), FIGS. 4(A) and 4(B) respectively show the Raman spectra for EMCV (10⁷ and 10⁶ PFU/mL) detection with the substrates Nos. 6 to 9. In FIGS. 4(A) and 4(B), the apAu denotes the detection performed on the substrate without cavity arrays and PBS (in #8) represents the detection performed with PBS containing no virus on the substrate No. 8.

Based on the results mentioned above, no virus signal is found on the substrate without cavity arrays, but EMCV signals are found in Raman shift ranging from 1100 to 1600 cm⁻¹. SERS spectrum for EMCV placed upon the substrate No. 8 exhibits the highest intensity and the bands of amino acids at 1558 and 1591 cm⁻¹ can be assigned to tryptophan and phenylalanine, respectively. Relatively minor band of amino acid at 1165 cm⁻¹ is assigned to tyrosine. In addition, Raman shift at 1232 cm⁻¹ is assigned to amide III, arising from the coupling of C-N stretching and N—H bonding, while the bands at 1302 and 1506 cm⁻¹ are also attributed to amide III (i.e., from protein) and N-H bending, respectively. As shown in FIGS. 4(A) and 4(B), Raman peaks in SERS spectra for EMCV detection on substrates Nos. 6 to 9 are all identical, regardless of EMCV concentrations in 10⁷ or 10⁶ PFU/ml. Above all, SERS intensity of EMCV detection performed on the substrate No. 8 (i.e., Dᵥ=90 nm) is the highest, which also corresponds well with the results of R6G molecule detection.

With reference to FIGS. 5(A) and 5(B), FIGS. 5(A) and 5(B) respectively show the Raman spectra for adenovirus (10⁷ and 10⁶ PFU/mL) detection with the substrates Nos. 7 to 9. In FIGS. 5(A) and 5(B), the apAu denotes the detection performed on the substrate without cavity arrays and PBS (in #8) represents the detection performed with PBS containing no virus on the substrate No. 8.

Based on the results mentioned above, no virus signal is found on the substrate without cavity arrays, but adenovirus signals are found in Raman shift ranging from 1200 to 1650 cm⁻¹. SERS spectrum for adenovirus placed upon the substrate No. 8 exhibits the highest intensity and the bands of amino acids at 1360 and 1591 cm⁻¹ can be assigned to tryptophan and phenylalanine, respectively. Raman shift at 1491 cm⁻¹ is attributed to C-N stretching vibration coupled with the in-plane C-H bending in amino radical cations. Furthermore, Raman shifts at 1650, 1302, and 1200 cm⁻¹ are assigned to amide I (i.e., from protein), amide III (i.e., from protein), and amide III (i.e., C-N stretching and N-H bending), respectively. Accordingly, the substrate No. 8 (i.e., Dᵥ=90 nm) is the most suitable one for adenovirus detection.

With reference to FIGS. 6(A) and 6(B), FIGS. 6(A) and 6(B) respectively show the Raman spectra for influenza A virus (10⁵ and 10⁴ PFU/mL) detection with the substrates Nos. 8 to 10. In FIGS. 6(A) and 6(B), the apAu denotes the detection performed on the substrate without cavity arrays and PBS (in #9) represents the detection performed with PBS containing no virus on the substrate No. 9.

Based on the results mentioned above, no virus signal is found on the substrate without cavity arrays, but influenza A virus signals are found in Raman shift ranging from 1100 to 1650 cm⁻¹ on the substrates Nos. 8 to 10. SERS spectrum for influenza A virus placed upon the substrate No. 9 exhibits the highest intensity and the bands of amino acids at 1360 and 1591 cm⁻¹ cm can be assigned to tryptophan and tyrosine, respectively. Raman shifts at 1165 and 1480 cm⁻¹ are assigned to amino acid of tyrosine and amide II (i.e., a coupling of C-N stretching and in-plane bending of the N-H group), respectively. Furthermore, Raman shift at 1260 cm⁻¹ was attributed to CH₂ in-plane deformation (i.e., from lipids on the envelope of influenza virus). Accordingly, the substrate No. 9 (i.e., Dᵥ=120 nm) is the most suitable one for influenza A virus detection.

It can be seen that different viruses can be easily distinguished on a chosen virus detection substrate when the enhanced signals of SERS effects can be obtained or Dᵥ can match the diameter of the tested virus, so as to give a qualitative result. In the tests mentioned above, the viruses are selected in sizes of 30, 90, and 120 nm. When the tested virus is in a size smaller than 90 nm, the substrate No. 8 can give optimized SERS effects since induced surface plasmon is most significant. When the tested virus is in a size more than 90 nm, the depth of the inverted triangular pyramid cavities can comply with the diameter of the tested virus, i.e. a size-depended effect so that SERS effects can be induced dramatically.

In another test, the EMCV, adenovirus, and influenza A virus solutions were pipetted in 5 μl on the substrates Nos. 8 and 9 and phosphotungstic acid (PTA, 2% PTA in 5 ml PBS) was simultaneously pipetted in 10 μl on a glass slide. After the virus solutions were dried on the virus detection substrates, the substrates were reversed to make the dried viruses become attached to PTA on the glass slide. Thus, the viruses were fixed on the substrates, then treated by negative staining for 2 minutes, washed twice with deionized water, and finally examined by field emission scanning electron microscope (FE-SEM, JSM-7001, JEOL, Japan).

The results are shown in FIG. 7. In FIG. 7, (A) denotes an FE-SEM photograph of EMCV; (B) denotes an FE-SEM photograph of adenovirus; and (C) denotes an FE-SEM photograph of influenza A virus. It can be seen that the inverted triangular pyramid cavities of the substrates Nos. 8 and 9 are like traps and thus EMCV and adenovirus are entrapped at the bottom tip of the cavities (see FIGS. 7(A) and 7(B)), or influenza A virus catches in the edge of the cavities (see FIG. 7(C)).

Based on the results illustrated above, the regulation of Dₜ₋ₜ and Dᵥ along with SERS technique is helpful to distinguish three viruses in various sizes, such as EMCV, adenovirus, and influenza A virus. Among three viruses, the detectable concentrations of EMCV and adenovirus are approximately 10⁶ PFU/ml and that of influenza A virus is approximately 10⁴ PFU/ml. Even though the tested virus is at different concentrations, the peak patterns of SERS spectra resulted from a virus detection substrate are substantially the same. Once the SERS spectrum database for various viruses is constructed, an unknown virus can be tested on the virus detection substrate by SERS spectrometry to get a specific spectrum, and the spectrum can be compared with the SERS spectra in the database, so as to identify the virus.

Although the present invention has been explained in relation to its preferred embodiment, it is to be understood that many other possible modifications and variations can be made without departing from the scope of the invention as hereinafter claimed.

What is claimed is:

1. A virus detection substrate used together with a Raman spectrometer for virus detection and comprising a metal layer disposed thereon, wherein multiple cavities are formed in the metal layer and arranged in a microarray.

2. The virus detection substrate as claimed in claim 1, wherein the metal layer is made of one selected from the group consisting of gold, silver, copper, and an alloy thereof.

3. The virus detection substrate as claimed in claim 1, wherein the cavities are formed in a shape of an inverted multi-angular pyramid.

4. The virus detection substrate as claimed in claim 3, wherein the distance between two of the cavities is in a range from 100 nm to 1000 nm, and the depth of the cavities is in a range from 50 nm to 300 nm.

5. A virus detection system used to detect a virus sample, comprising:
a virus detection substrate used to hold the virus sample and comprising a metal layer disposed thereon, wherein multiple cavities are formed in the metal layer and arranged in a microarray;
a Raman spectrometer applying an incident laser onto the metal layer of the virus detection substrate to obtain a Raman scattering signal; and
a receiver device used to receive the Raman scattering signal and output a Raman spectrum.

6. The virus detection system as claimed in claim 5, wherein the metal layer is made of one selected from the group consisting of gold, silver, copper, and an alloy thereof.

7. The virus detection system as claimed in claim 5, wherein the cavities are formed in a shape of an inverted multi-angular pyramid.

8. The virus detection system as claimed in claim 7, wherein the distance between two of the cavities is in a range from 100 nm to 1000 nm, and the depth of the cavities is in a range from 50 nm to 300 nm.

9. A method for detecting viruses, comprising the following steps:
providing a virus detection substrate and a Raman spectra virus database, wherein the virus detection substrate comprises a metal layer disposed thereon and multiple cavities are formed in the metal layer and arranged in a microarray;
dropping a virus sample onto the inner surface of the cavities of the virus detection substrate;
applying an incident light by a Raman spectrometer onto the metal layer of the virus detection substrate to generate a Raman spectrum of the virus sample; and
comparing the Raman spectrum of the virus sample with the Raman spectra virus database to identify the species of the virus sample.

10. The method as claimed in claim 9, wherein the metal layer is made of one selected from the group consisting of gold, silver, copper, and an alloy thereof.

11. The method as claimed in claim 9, wherein the cavities are formed in a shape of an inverted multi-angular pyramid.

12. The method as claimed in claim 11, wherein the distance between two of the cavities is in a range from 100 nm to 1000 nm, and the depth of the cavities is in a range from 50 nm to 300 nm.
